(19) United States
(12) Patent Application Publication
(10) Pub. No.: US 2026/0273206 A1
(43) Pub. Date: Sep. 17, 2026

(54) PERFORATED INHALATION MASK

(71) Applicant: Fridababy, LLC, Miami, FL (US)

(72) Inventors: Monica TOURNOUX, Miami, FL (US); Sophie FORNARO, Miami, FL (US); Chelsea HIRSCHHORN, Miami Beach, FL (US)

(21) Appl. No.: 19/080,214

(22) Filed: Mar. 14, 2025

(51) Int. Cl. A61M 15/00

(52) U.S. Cl. CPC A61M 15/009

(57) ABSTRACT

A nebulizer mask includes a body defining an interior chamber for receiving aerosolized medication, the body having an interior surface and an exterior surface. The nebulizer mask also includes a connector interface, at a tip of the mask, for attaching the nebulizer mask to a nebulizer device. The nebulizer mask further includes a group of perforations on the body, each perforation penetrating the interior surface and the exterior surface.

### BACKGROUND

### Field

Aspects of the present disclosure generally relate to medical devices for respiratory therapy, and more specifically to a perforated inhalation mask for respiratory therapy.

### Background

Nebulizers may be used to administer inhaled substances, such as medicine, for treating respiratory conditions, such as, but not limited to, asthma, chronic obstructive pulmonary disease (COPD), bronchitis, and cystic fibrosis. A nebulizer device converts liquid medication into a fine mist or aerosol, allowing a patient to inhale the medication directly into their upper and lower respiratory tracts. The nebulized medication is typically delivered via a face mask or mouthpiece, with face masks being desirable for young children, elderly patients, and individuals with difficulty using a mouthpiece.

### SUMMARY

In some aspects, a nebulizer mask includes a body defining an interior chamber for receiving aerosolized medication, the body having an interior surface and an exterior surface. The nebulizer mask also includes a connector interface, at a tip of the mask, for attaching the nebulizer mask to a nebulizer device. The nebulizer mask further includes a group of perforations on the body, each perforation penetrating the interior surface and the exterior surface.

Other aspects are directed to a method for delivering aerosolized medication using a nebulizer mask. The method includes attaching the nebulizer mask to a nebulizer device. The method also includes directing the aerosolized mist into an interior chamber of a nebulizer mask, the mask having a group of perforations for allowing mixing of the aerosolized mist with ambient air. The method further includes inhaling the aerosolized mist through the nebulizer mask, the group of perforations regulating airflow and medication concentration.

This has outlined, rather broadly, the features and technical advantages of the present disclosure in order that the detailed description that follows may be better understood. Additional features and advantages of the present disclosure will be described below. It should be appreciated by those skilled in the art that this present disclosure may be readily utilized as a basis for modifying or designing other structures for carrying out the same purposes of the present disclosure. It should also be realized by those skilled in the art that such equivalent constructions do not depart from the teachings of the present disclosure as set forth in the appended claims. The novel features, which are believed to be characteristic of the present disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

The features, nature, and advantages of the present disclosure will become more apparent from the detailed description set forth below when taken in conjunction with the drawings in which like reference characters identify correspondingly throughout.

FIG. 1 is a diagram illustrating an example of a nebulizer system comprising a nebulizer and a perforated nebulizer mask, in accordance with various aspects of the present disclosure.

FIG. 2 is a diagram illustrating an exploded view of a connection between a perforated nebulizer mask and a nebulizer, in accordance with various aspects of the present disclosure.

FIGS. 3A, 3B, 3C, 3D, 3E, 4A, 4B, 5A, 5B, 5C, 5D, 5E, 6A, 6B, and 6C illustrate examples of a perforated nebulizer mask, in accordance with various aspects of the present disclosure

FIG. 7 is a flow diagram illustrating an example of a process for using a perforated nebulizer mask, in accordance with various aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. It will be apparent to those skilled in the art, however, that these concepts may be practiced without these specific details. In some instances, well-known structures and components are shown in block diagram form in order to avoid obscuring such concepts.

Based on the teachings, one skilled in the art should appreciate that the scope of the present disclosure is intended to cover any aspect of the present disclosure, whether implemented independently of or combined with any other aspect of the present disclosure. For example, an apparatus may be implemented, or a method may be practiced using any number of the aspects set forth. In addition, the scope of the present disclosure is intended to cover such an apparatus or method practiced using other structure, functionality, or structure and functionality in addition to, or other than the various aspects of the present disclosure set forth. It should be understood that any aspect of the present disclosure may be embodied by one or more elements of a claim.

The word “exemplary” is used herein to mean “serving as an example, instance, or illustration.” Any aspect described herein as “exemplary” is not necessarily to be construed as preferred or advantageous over other aspects.

Although particular aspects are described herein, many variations and permutations of these aspects fall within the scope of the present disclosure. Although some benefits and advantages of the preferred aspects are mentioned, the scope of the present disclosure is not intended to be limited to particular benefits, uses, or objectives. Rather, aspects of the present disclosure are intended to be broadly applicable to different technologies, system configurations, networks, and protocols, some of which are illustrated by way of example in the figures and in the following description of the preferred aspects. The detailed description and drawings are merely illustrative of the present disclosure rather than limiting, the scope of the present disclosure being defined by the appended claims and equivalents thereof.

As discussed, nebulizers may be used to administer inhaled substances, such as medicine, for treating respiratory conditions, such as, but not limited to, congestion, asthma, chronic obstructive pulmonary disease (COPD), bronchitis, and cystic fibrosis. A nebulizer device converts liquid medication into a fine mist or aerosol, allowing a patient to inhale the medication directly into their upper and lower respiratory tracts. The nebulized medication is typically delivered via a face mask or mouthpiece, with face masks being preferred for young children, elderly patients, and individuals with difficulty using a mouthpiece.

A nebulizer mask is an example of a mask for the administration of medicine, or another substance, via a nebulizer. Nebulizer masks may also be referred to as aerosol masks. Conventional nebulizer masks function as sealed enclosures that cover a patient’s nose and mouth, forming a confined space where aerosolized medication accumulates before inhalation. While this design provides for controlled medication delivery, the sealed enclosure design presents several efficiency and comfort challenges. One of the primary issues is poor air mixing, where the concentration of aerosolized medication fluctuates throughout the inhalation process. This results in higher doses in the initial breaths and weaker concentrations over time, leading to inconsistent drug delivery. Additionally, these enclosed masks contribute to medication condensation and waste, as a portion of the aerosolized medication condenses on the inner surface before being inhaled. This reduces the actual dose received by the patient, decreasing the overall therapeutic effectiveness of the treatment.

Another limitation of conventional nebulizer masks is high inhalation resistance. The enclosed design creates increased airflow resistance, making it more difficult for pediatric, elderly, or respiratory-compromised patients to breathe comfortably. Patients with weakened lung function may struggle to draw in sufficient medication, further reducing the mask’s effectiveness. Additionally, leakage and ineffective sealing are common issues, particularly in children who may not maintain a proper fit. A poor seal leads to medication escaping the mask, causing wasted doses and reduced treatment efficiency.

Various aspects of the present disclosure relate to a perforated nebulizer mask designed to improve air mixing, providing a consistent concentration of medication in each breath while reducing condensation to reduce waste and improve drug delivery efficiency. The perforated nebulizer mask includes one or more perforations, such as holes and/or slits. The one or more perforations control an amount of ambient air that is mixed with the medicine, thereby reducing inhalation resistance and making it easier for patients to inhale the medication. Additionally, the perforated nebulizer mask provides a secure yet comfortable fit, minimizing condensation, leakage and drug loss.

Particular aspects of the subject matter described in this disclosure can be implemented to realize one or more of the following potential advantages. In some examples, the described techniques, such as the incorporation of perforations in the nebulizer mask, may improve air mixing, reduce condensation, and optimize drug delivery efficiency. Additionally, these techniques may improve breathability by allowing controlled ambient air mixing, thereby reducing inhalation resistance. In some implementations, the design may provide a more secure fit, minimizing medication leakage and drug loss. Furthermore, the perforated mask structure may improve patient comfort by reducing the effort required for inhalation, making it particularly beneficial for pediatric, elderly, and respiratory-compromised patients.

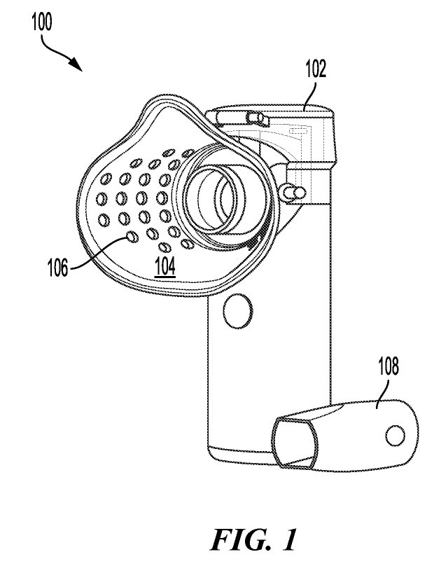

FIG. 1 is a diagram illustrating an example of a nebulizer system 100 comprising a nebulizer 102 and a perforated nebulizer mask 104, in accordance with various aspects of the present disclosure. As shown in the example of FIG. 1, multiple holes 106 are integrated into a structure of the mask 104. The holes 106 are examples of perforations. Aspects of the present disclosure are not limited to using holes 106 as perforations, other types of perforations, such as slits, are contemplated. In the current applications, holes 106 and perforations may be used interchangeably. Still, the perforations are not limited to holes.

The nebulizer 102 is an example of a portable medical device designed to convert liquid medication or another liquid substance into an inhalable aerosol mist, which is then delivered to the patient through the perforated nebulizer mask 104. For ease of explanation, the perforated nebulizer mask 104 may be referred to as a nebulizer mask 104 or a mask 104 (hereinafter used interchangeably). The mask 104 is specified to cover a patient’s nose and mouth, forming a semi-enclosed chamber where the aerosolized medication can mix with ambient air before being inhaled. In contrast to conventional nebulizer masks that rely on a fully enclosed chamber, the perforated nebulizer mask 104 incorporates strategically placed holes 106, which facilitate controlled ambient air mixing to improve breathability and drug delivery efficiency. Specifically, the holes 106 may be placed in specific locations on the mask 104 to balance medication containment and airflow optimization, thus reducing the inhalation resistance typically associated with fully enclosed masks.

The nebulizer 102 operates by atomizing liquid medication into fine aerosolized particles that can be inhaled directly into the patient’s lungs. The nebulizer 102 may utilize one of several atomization mechanisms, including jet nebulization, ultrasonic nebulization, or vibrating mesh technology. In a jet nebulizer configuration, compressed air is forced through a narrow orifice, creating a vacuum that draws liquid medication from a reservoir and breaks the liquid into fine droplets. In an ultrasonic nebulizer, high-frequency sound waves agitate the liquid, producing aerosolized particles. In a vibrating mesh nebulizer, a perforated membrane oscillates at high frequency, pushing liquid medication through microscopic holes to generate a fine mist. Regardless of the specific technology used, the generated aerosol is delivered to the perforated nebulizer mask 104, where it is inhaled by the patient.

As discussed, in contrast to conventional nebulizer masks that rely on a fully enclosed structure to contain aerosolized medication, the perforated nebulizer mask 104 features holes 106 distributed across its surface. These holes 106, which constitute at least ten percent of the mask’s total surface area, are positioned to allow controlled ambient air mixing, thereby reducing the effort required for inhalation and minimizing medication condensation inside the mask. The size, shape, and distribution of the holes 106 may be adjusted to control the degree of air mixing, improving consistency in medication delivery. In one embodiment, the holes 106 may be uniformly spaced to provide an even distribution of ambient air across the entire mask surface. In another embodiment, the perforations may be non-uniform, with smaller holes near the center of the mask, closer to the patient’s nose and mouth, and larger holes near the periphery, allowing for a gradual air-mixing gradient that optimizes medication dispersion.

The perforated nebulizer mask 104 may be constructed from medical-grade, hypoallergenic materials, such as silicone, PVC, or thermoplastic elastomers, to improve comfort and durability for extended use. In one embodiment, the mask 104 may include an antimicrobial coating to reduce bacterial contamination, particularly for patients who require frequent nebulizer treatments. Additionally, the mask 104 may be designed to be flexible and form-fitting, providing a secure yet comfortable fit that minimizes medication leakage while still allowing for unrestricted breathing.

In some embodiments, the diffuser attachment 108 may be included as an optional component to further modify the characteristics of the aerosolized medication before inhalation. Specifically, the diffuser attachment 108 is a component that may be affixed to the nebulizer 102 to modify airflow patterns or adjust the dispersion characteristics of the aerosolized medication. The diffuser attachment 108 may be used instead of the mask 104. In some examples, diffuser attachment 108 may function to control particle size distribution, allowing the medication to reach the appropriate regions of the lungs. In one embodiment, the diffuser attachment 108 may contain a flow regulator that adjusts the velocity of the aerosol mist, allowing for more efficient drug deposition in the lower respiratory tract. In another embodiment, the diffuser attachment 108 may include a humidity-enhancing element, such as a small integrated water reservoir or membrane, to add moisture to the inhaled medication, reducing throat irritation and improving patient comfort.

In some examples, the perforated nebulizer mask 104 may include adjustable perforation settings, where the size and number of holes 106 can be manually or automatically modified based on the patient’s breathing capacity and prescribed treatment parameters. In one such example, the mask 104 may incorporate a sliding perforation cover that allows the user to increase or decrease the number of active perforations, adjusting the degree of air mixing. In another example, the mask may include one-way valves that control the direction of airflow, allowing for a more customized breathing experience.

The perforated nebulizer mask 104 is designed to be compatible with a variety of nebulizer 102 models, including jet nebulizers, vibrating mesh nebulizers, and ultrasonic nebulizers. A connector interface of the mask 104 may be standardized to accommodate different nebulizer designs, making the mask 104 versatile and adaptable for various patient needs. In another example, the connector interface may be interchangeable or adaptable to attach to different nebulizer designs. The mask 104 may be available in multiple sizes, including infant, pediatric, and adult versions, to improve fit for different patient demographics.

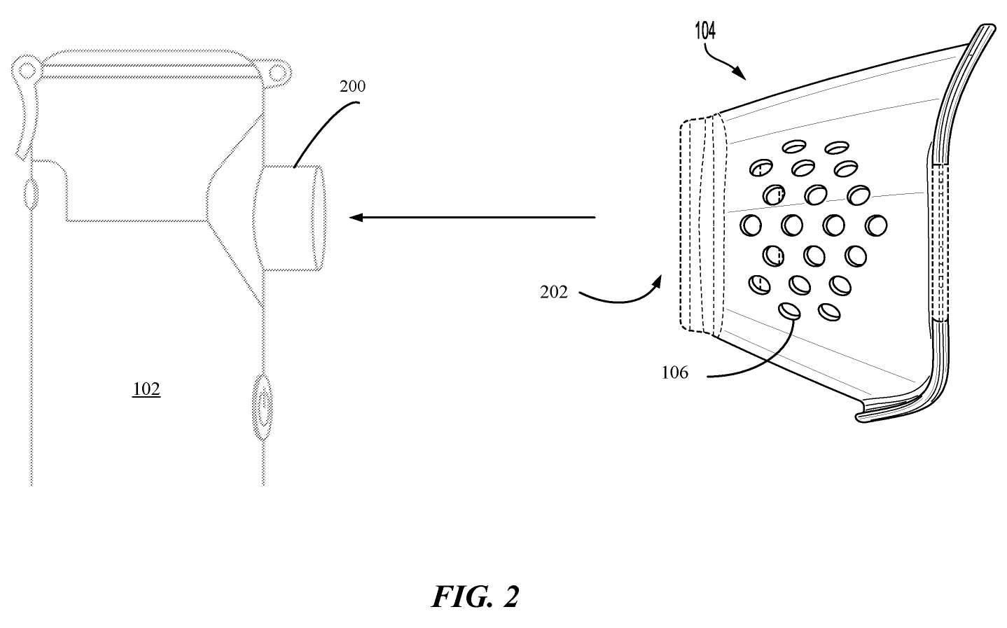

FIG. 2 is a diagram illustrating an exploded view of a connection between a perforated nebulizer mask 104 and a nebulizer 102, in accordance with various aspects of the present disclosure. The perforated nebulizer mask 104 is designed to deliver aerosolized medication to a patient while allowing controlled ambient air mixing through multiple holes 106 distributed across its surface. The nebulizer 102 generates the aerosolized medication, which is then directed through a connector 200 to a connector interface 202 of the perforated nebulizer mask 104. The connector interface 202 may be a cylindrical opening at a tip of the mask 104. In the example of FIG. 2, the connector interface 202 is shown with dashed lines to exemplify that the structure of the connector interface 202 shown in the example of FIG. 2 is optional. Other types of connector interfaces may be used. In some examples, the connector interface 202 may be interchangeable or re-configurable.

The connector 200 of the nebulizer 102 serves as the attachment point for the perforated nebulizer mask 104. The connector interface 202 of the perforated nebulizer mask 104 is structured to securely fit onto the connector 200, creating a stable fluid connection that allows aerosolized medication to flow efficiently from the nebulizer 102 into the mask. The connector interface 202 may include various attachment mechanisms to improve compatibility with different nebulizer models. In one embodiment, the connector interface 202 may feature a friction-fit coupling, allowing the mask 104 to slide onto the connector 200 and be held in place by compression. In another embodiment, the connector interface 202 may use a threaded connection, where the mask 104 is twisted onto the connector 200 to create a secure fit. Alternatively, the connector interface 202 may employ a snap-fit or locking mechanism, ensuring that the mask 104 remains attached to the nebulizer 102 during use, even if the patient moves or adjusts their positioning.

The holes 106 in the perforated nebulizer mask 104 are strategically placed to allow controlled ambient air mixing, reducing inhalation resistance while maintaining an effective concentration of aerosolized medication. The size, number, and distribution of the holes 106 can be customized for different patient needs. In one embodiment, the holes 106 may be symmetrically arranged to provide uniform air-mixing properties across the mask. In another embodiment, the holes 106 may vary in size and density, with smaller holes located near the center of the mask for higher medication concentration and larger holes near the edges to allow gradual ambient air mixing.

The perforated nebulizer mask 104 may be constructed from flexible, medical-grade materials such as silicone, PVC, or thermoplastic elastomers. These materials provide a comfortable fit while ensuring a secure connection between the mask and the nebulizer. Additionally, in some embodiments, the mask 104 may include an antimicrobial coating, reducing contamination risks for patients requiring frequent nebulization therapy.

The connector interface 202 may be designed to accommodate multiple nebulizer types, including jet nebulizers, ultrasonic nebulizers, and vibrating mesh nebulizers. In one embodiment, the connector interface 202 may include a universal adapter, allowing the perforated nebulizer mask 104 to be used with different nebulizer models without requiring additional components. In another embodiment, the connector interface 202 may have an interchangeable fitting system, where patients can swap out different adapters based on their specific nebulizer type. The design of the connector 200 and connector interface 202 allows for a secure and stable attachment, such that the aerosolized medication is delivered to the patient while minimizing leaks and loss of medication.

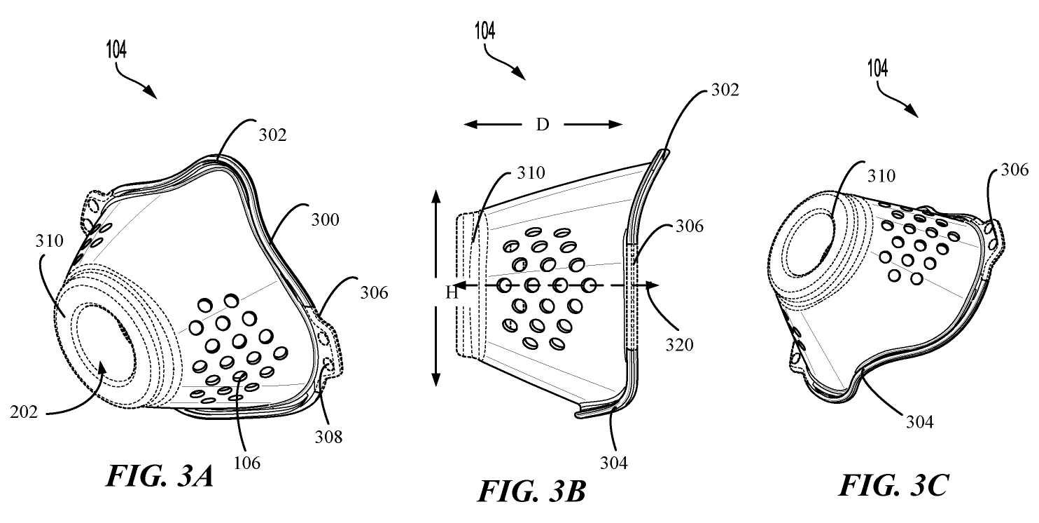

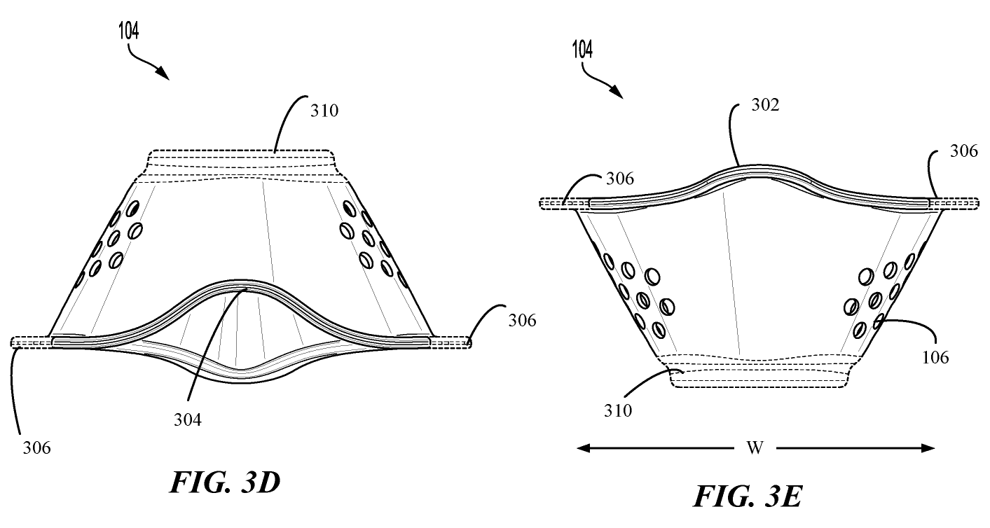

FIGS. 3A, 3B, 3C, 3D, and 3E illustrate examples of a perforated nebulizer mask 104, in accordance with various aspects of the present disclosure. For brevity, some elements may not be numbered in each one of FIGS. 3A, 3B, 3C, 3D, and 3E. FIG. 3A illustrates an example of a top-right perspective view of the mask 104, FIG. 3B illustrates an example of a side view, FIG. 3C illustrates an example of a bottom-right perspective view, FIG. 3D illustrates an example of a bottom view, and FIG. 3E illustrates an example of a top view.

As shown in FIGS. 3A, 3B, 3C, 3D, and 3E, the perforated nebulizer mask 104 may have a contoured shape around an outer perimeter 300 that conforms to the user’s face, providing a comfortable and stable fit. The mask 104 expands outward in both a vertical plane and a horizontal plane from a tip 310. A connector interface 202 is defined at the tip 310. As shown in the example of FIGS. 3A and 3C, the connector interface 202 is a cylindrical opening within the tip that extends from a tip toward an inner area (e.g., chamber) of the mask 104. The cylindrical opening is hollow to create a channel that allows the medicine to enter the chamber of the mask 104.

Additionally, as shown in FIGS. 3A, 3B, 3C, 3D, and 3E, an apex 302 of the outer perimeter 300 flares toward the user’s face to cover the nose, while a bottom 304 of the outer perimeter 300 is concave to accommodate an area below the user’s mouth, creating a seal. The ergonomic curvature of the outer perimeter 300 is designed to align with the nose and mouth, allowing for efficient inhalation of aerosolized medication. The outer perimeter 300 may include a soft lip that extends outward from the interior of the mask 104. Additionally, at each distal lateral end of the vertical plane, tabs 306 may be formed to accommodate a strap. Each tab 306 may include one or more holes 308 for securing a strap.

The mask 104 may have a height H, as shown in FIG. 3B, of about 65 mm. In some examples, the height is 65.4 mm. The depth D, also shown in FIG. 3B, may be about 50 mm. In some examples, the depth is 50.9 mm. The height may be measured from the bottom-most portion of the mask 104 in a horizontal plane to the apex 302. The depth may be measured from the tip of the mask 104 at the connector interface 202 to the tip of the outer perimeter 300 in a horizontal plane. The width W, as shown in FIG. 3E, may be about 78 mm, with some variations where the width is 78.9 mm. The width may be measured from one end of a tab 306 to the opposite tab 306. If the mask 104 does not include tabs 306, the width may be measured from one lateral end of the mask 104 to the other lateral end. Other measurements for the height, depth, and width are contemplated and are not limited to the specified dimensions.

As shown in FIGS. 3A through 3E, the mask 104 incorporates a structured arrangement of forty holes 106, with twenty holes 106 on each lateral side of the mask 104. Aspects of the present disclosure are not limited to twenty holes 106 on each later side, additional or fewer holes may be used. The holes 106 may be positioned to improve air mixing by allowing controlled inflow of ambient air while minimizing medication condensation inside the mask. The holes 106 collectively cover at least ten percent of the total surface area of the mask 104.

The layout of the holes 106 follows a structured pattern. As shown in FIG. 3B, a first set of holes 106 are defined along a central horizontal axis 320 from the tip 310 of the mask 104, toward the face-covering portion that rests against the user. The central horizontal axis 320 may also be referred to as a central axis 320 or an axis 320. Other holes 106 are distributed on either side of the axis 320 in a mirrored configuration, ensuring balanced air intake. The other holes 106 are placed along horizontal axes that are parallel to the central horizontal axis 320. In some examples, a number of holes 106 along the central horizontal axis 320 is greater than a number of holes 106 along each horizontal axis that are parallel to the central horizontal axis 320.

In the examples of FIGS. 3A through 3E, a first set and second set of holes 106 is defined on each side of the mask 104. The holes 106 along the central axis 320 may be a first subset of holes 106 from both the first and second set of holes 106. Additional subsets of holes 106, such as an additional subset 350 shown in the example of FIG. 3B, are arranged along respective horizontal axes, extending outward from the center horizontal axis 320. Each of these additional subsets of holes 106 is positioned parallel to one of the first subset of perforations 106. In some examples, the additional subset of holes are not distributed in a mirrored configuration respective to the central horizontal axis 320.

In some examples, all holes 106 have the same diameter to maintain uniform airflow distribution throughout the mask 104. In other examples, the diameter of the holes 106 gradually increases toward the outer portions of the mask 104 to create a controlled air-mixing gradient. In yet another embodiment, the holes 106 may be arranged in alternating rows of different diameters to allow for differential airflow, promoting improved medication retention while maintaining lower inhalation resistance. The mirrored arrangement of the holes 106 on both lateral sides of the mask 104 allows for consistent air-mixing effects. In some examples, the arrangement of the holes 106 may be asymmetrical.

In some embodiments, the mask 104 may include adjustable holes 106, where the holes 106 can be opened or closed using a sliding cover mechanism. This design may allow a caregiver or patient to regulate the amount of ambient air mixing based on breathing capacity and medication concentration requirements. In another embodiment, the holes 106 may be equipped with one-way airflow valves, permitting ambient air to enter while preventing medication loss through exhalation.

The mask 104 may also feature a multi-layer construction, where an inner layer is made of a softer material to improve comfort, and an outer layer is more rigid to maintain structural integrity. In one embodiment, the mask 104 may include an antimicrobial coating to reduce bacterial contamination, particularly for patients who require frequent nebulization therapy. In another embodiment, the material of the mask 104 may be heat-resistant to allow for sterilization without deformation.

The mask 104 also includes a central connector interface 202 designed for attachment to a nebulizer device. In some examples, the connector interface 202 is cylindrical and includes a smooth interior surface to provide a secure and airtight connection to the nebulizer’s outlet. In other embodiments, the connector interface 202 may include a threaded attachment or a locking mechanism to improve stability and compatibility with various nebulizer models. In one example, the connector interface 202 may incorporate an integrated flow regulator, allowing precise control of medication flow rates. Other connector interface configurations are contemplated and are not limited to the example shown in FIGS. 3A through 3E.

In some embodiments, the mask 104 may be provided in multiple sizes to accommodate different patient demographics, including pediatric, adult, and specialized medical-use versions. In another embodiment, the mask 104 may feature a removable face-seal attachment, allowing for further customization of the fit.

In the examples of FIGS. 3A, 3B, 3C, 3D, and 3E, the tip 310, connector interface 202, and the tabs 306 are shown with dashed lines to exemplify that the structure of the tip 310, the connector interface 202, and tabs 306 shown in the examples of FIGS. 3A, 3B, 3C, 3D, and 3E are optional. Other types of connector interfaces or tabs may be used. In some examples, the tip 310 and/or the connector interface 202 may be interchangeable or re-configurable.

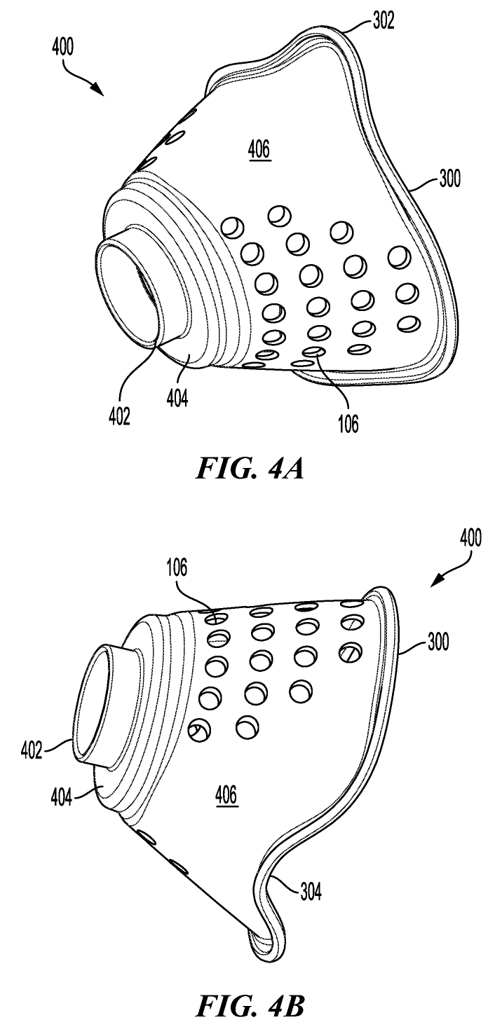

FIGS. 4A and 4B illustrate examples of a perforated nebulizer mask 400, in accordance with various aspects of the present disclosure. Various elements of the mask 400 shown in FIG. 4 are similar to the mask 104 described with reference to FIGS. 1, 2, 3A, 3B, 3C, 3D, and 3E. For brevity, a description of the similar elements will be omitted from the description of the mask 400 shown in FIGS. 4A and 4B. In contrast to the mask 104 described with reference to FIGS. 1, 2, 3A, 3B, 3C, 3D, and 3E, the mask 400 shown in FIGS. 4A and 4B does not include tabs at distal lateral ends. Still, in some examples, the mask 400 may include tabs at distal ends. Additionally, in the example of FIGS. 4A and 4B, the mask 400 includes a cylindrical connector interface 402 that extends from a tip 404 of the mask 400. In such examples, a diameter of the cylindrical connector interface 402 is less than a diameter of the tip 404 of the mask 400. The tip 404 of the mask 104 is a flat surface that serves as the transition point between the main body 406 of the mask 400 and the cylindrical connector interface 402

Extending outward from the flat tip 404, the cylindrical connector interface 402 is designed to accommodate a nebulizer outlet securely. The cylindrical shape may provide a stable attachment mechanism, reducing the likelihood of unintended disconnection during use. The outer surface of the cylindrical connector interface 402 may include one or more concentric ridges or grooves that may enhance grip and improve sealing efficiency when the mask 400 is connected to a nebulizer.

In some examples, the cylindrical connector interface 402 may include an interior taper, allowing it to accommodate nebulizer outlets of varying diameters while maintaining a snug fit. This taper may facilitate a friction-fit connection, ensuring that the nebulizer remains properly engaged with the mask without requiring additional fastening components. In other embodiments, the cylindrical connector interface 402 may incorporate a locking mechanism, such as a twist-lock or snap-fit system, to provide added stability and prevent accidental detachment during nebulization.

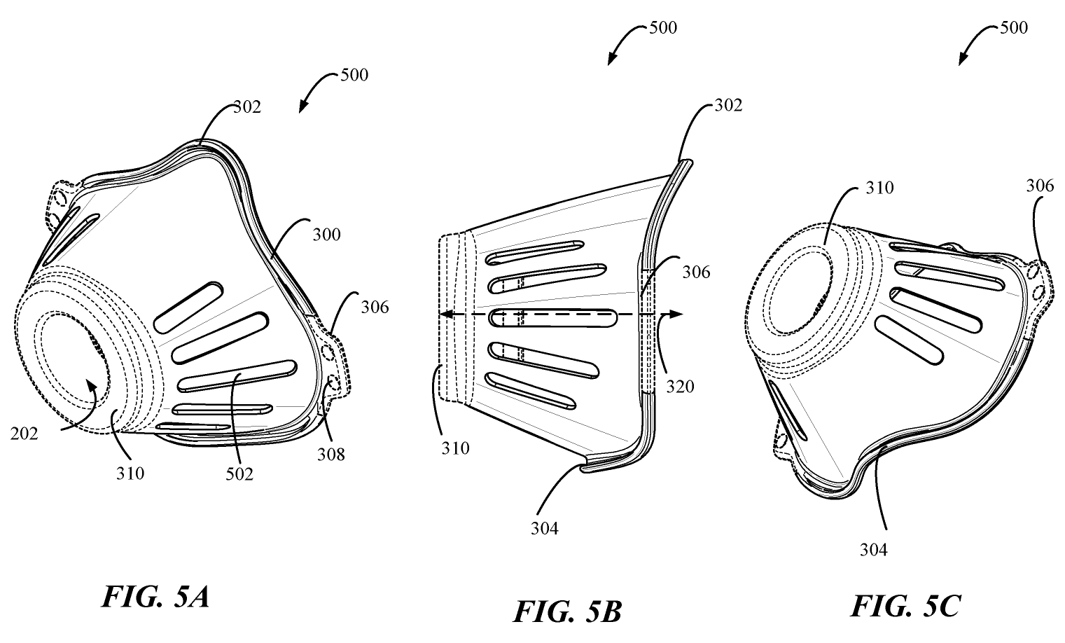

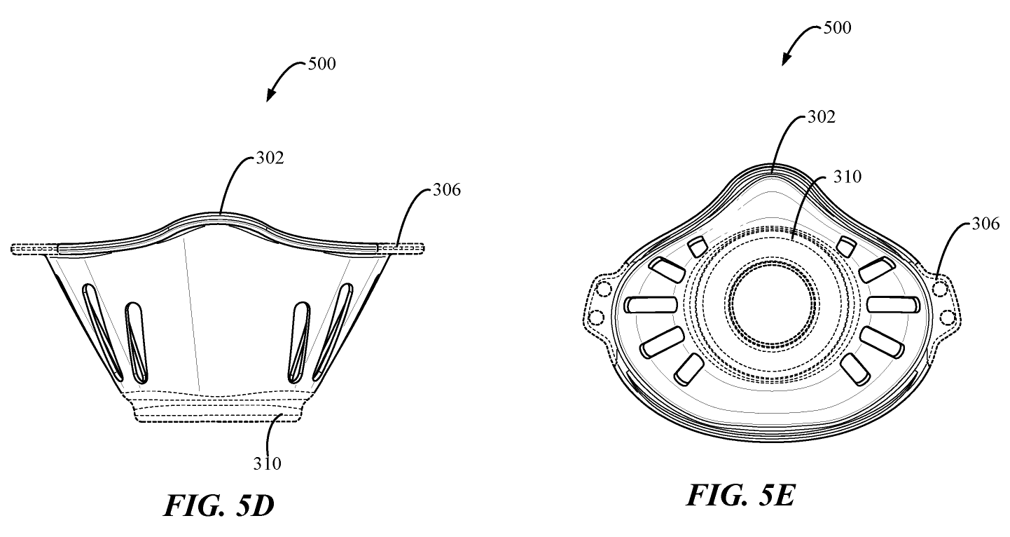

FIGS. 5A, 5B, 5C, 5D, and 5E illustrate examples of a perforated nebulizer mask 500, in accordance with various aspects of the present disclosure. For brevity, some elements may not be numbered in each one of FIGS. 5A, 5B, 5C, 5D, and 5E. FIG. 5A illustrates an example of a top-right perspective view of the mask 104, FIG. 5B illustrates an example of a side view, FIG. 5C illustrates an example of a bottom-right perspective view, FIG. 5D illustrates an example of a bottom view, and FIG. 5E illustrates an example of a front facing view. Various elements of the mask 500 shown in FIGS. 5A, 5B, 5C, 5D, and 5E are similar to the mask 104 described with reference to FIGS. 1, 2, 3A, 3B, 3C, 3D, and 3E. For brevity, a description of the similar elements will be omitted from the description of the mask 500 shown in FIGS. 5A, 5B, 5C, 5D, and 5E.

As shown in the examples of FIGS. 5A, 5B, 5C, 5D, and 5E, the mask 500 incorporates a structured arrangement of elongated slits 502 rather than circular perforations. These slits 502 are positioned to improve air mixing by allowing a controlled inflow of ambient air while minimizing medication condensation inside the mask. The slits 502 collectively cover at least ten percent of the total surface area of the mask 500.

The layout of the slits 502 follows a structured and symmetrical pattern. As shown in FIG. 5B, a first slit 502, of the group of slits 502 on each lateral side, is positioned along the central axis 320, running horizontally across the central region of the mask 500. Beyond the first slit 502, additional slits 502 are placed at an angle relative to the central axis 320, extending along a body of the mask 500 in both the upper and lower regions of the mask 500. These slits 502 are arranged in a symmetrical pattern on both lateral sides of the mask 500. The angled placement of the slits 502 directs the airflow in a controlled manner, allowing for gradual air mixing while reducing turbulence inside the mask. The orientation of the slits 502 ensures that the aerosolized medication remains evenly distributed as the patient inhales.

On each lateral side of the mask 500, the group of slits 502 are positioned in a staggered arrangement, with each slit 502 separated by a uniform spacing. These slits 502 extend from a region near the tip 310 toward the outer perimeter 300. The parallel alignment of the slits 502 facilitates a controlled air-mixing effect by allowing ambient air to enter in a uniform direction.

In some examples, the slits 502 may have a uniform length across the mask 500. In other examples, the first slit 502 along the central axis 320 may be the longest, with the slits 502 positioned on either side of it gradually decreasing in length as they are positioned away from the first slit 502. Each successive slit 502, in either direction away from the first slit 502, may be shorter than the one preceding it, creating a tapered arrangement.

Additionally, in some examples, the slits 502 may be adjustable, enabling a user to open or close specific slits based on individual treatment needs. This adjustability may allow for customized airflow regulation, optimizing the balance between medication retention and breathability. Additionally, or alternatively, in some examples, a height of each slit 502 may vary. In other examples, all slits 502 may have a uniform height. In still other examples, the height of each slit 502 may taper, either decreasing from the tip 310 toward the outer perimeter 300 or increasing in the opposite direction. This variation in slit height may further refine airflow characteristics, influencing the rate and distribution of ambient air mixing with the aerosolized medication. Additionally, or alternatively, in some examples, the slits 502 may be equipped with flexible membranes or one-way airflow valves that allow air to enter while preventing medication loss through exhalation. In another embodiment, the slits 502 may have reinforced edges to maintain their shape over extended use, providing durability and consistent performance.

In the examples of FIGS. 5A, 5B, 5C, 5D, and 5E, the tip 310, the connector interface 202, and the tabs 306 are shown with dashed lines to exemplify that the structure of the tip 310, the connector interface 202 and tabs 306 shown in the examples of FIGS. 5A, 5B, 5C, 5D, and 5E are optional. Other types of connector interfaces or tabs may be used. In some examples, the tip 310 and/or the connector interface 202 may be interchangeable or re-configurable.

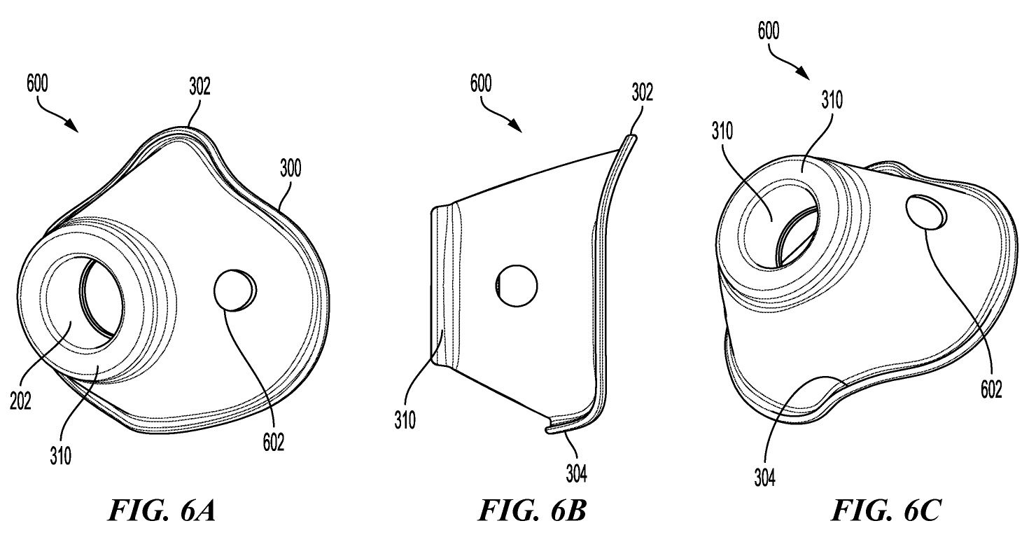

FIGS. 6A, 6B, and 6C illustrate examples of a perforated nebulizer mask 600, in accordance with various aspects of the present disclosure. For brevity, some elements may not be numbered in each one of FIGS. 6A, 6B, and 6C. FIG. 6A illustrates an example of a top-right perspective view of the mask 104, FIG. 6B illustrates an example of a side view, and FIG. 6C illustrates an example of a bottom-right perspective view. Various elements of the mask 600 shown in FIGS. 6A, 6B, and 6C are similar to the mask 104 described with reference to FIGS. 1, 2, 3A, 3B, 3C, 3D, and 3E. For brevity, a description of similar elements will be omitted from the description of the mask 600 shown in FIGS. 6A, 6B, and 6C.

In contrast to the mask 104 described with reference to FIGS. 1, 2, 3A, 3B, 3C, 3D, and 3E, the mask 600 shown in FIGS. 6A, 6B, and 6C does not include tabs at distal lateral ends. Instead, the outer perimeter 300 of the mask 600 forms a continuous structure without additional mounting extensions, such as strap attachment tabs. However, in some examples, the mask 600 may include tabs at distal ends similar to those in the mask 104 to provide additional options for securing the mask to the user’s face. It should be noted that the outer perimeter 300 of the mask 104 described with reference to FIGS. 1, 2, 3A, 3B, 3C, 3D, 3E, 5A, 5B, 5C, 5D, and 5E may also include a continuous structure without additional mounting extensions, such as strap attachment tabs.

As shown in the examples of FIGS. 6A, 6B, and 6C, the mask 600 includes a substantially circular opening 602 on each lateral side of the mask 600. As shown in FIGS. 6A, 6B, and 6C, these perforations 602 are symmetrically positioned and spaced away from the central region of the mask. The placement of these circular openings 602 allows for controlled air mixing while reducing the overall number of openings compared to prior designs.

The size and positioning of the circular openings 602 may be optimized to balance airflow resistance and medication retention within the mask 600. In some examples, the circular openings 602 may have a uniform diameter. In other embodiments, the circular openings 602 may have a non-uniform diameter or a non-circular shape.

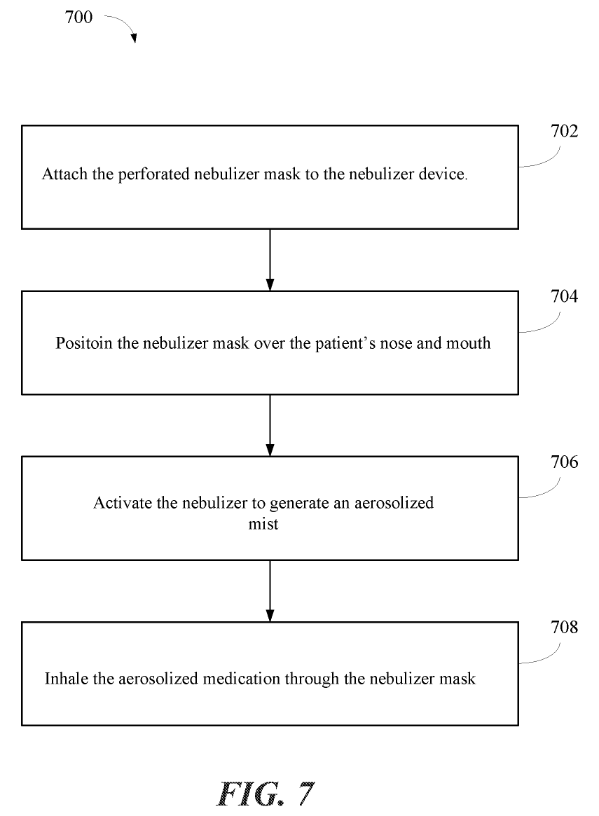

FIG. 7 is a flow diagram illustrating an example of a process 700 for using a perforated nebulizer mask, in accordance with various aspects of the present disclosure. The nasal suction device may be an example of a perforated nebulizer mask 104, 500, or 600, described with reference to FIGS. 1 through 6C. The process begins at block 702 by attaching the perforated nebulizer mask to the nebulizer device. This may involve securing the mask’s connector interface to the nebulizer’s outlet. As discussed, the connector interface may be attached via one or more mechanisms, such as, but not limited to, a friction-fit, threaded, or locking mechanism to provide a secure and stable connection. The user may also inspect the mask to confirm that the perforations (e.g., slits 502, openings 602) are unobstructed and positioned for optimal air mixing.

At block 704, the user positions the nebulizer mask over the patient’s nose and mouth. The mask should create a comfortable seal against the face, ensuring effective delivery of aerosolized medication. If the mask includes adjustable straps, the user may fasten or tighten the straps for a secure fit. The user may be the patient, such that the user positions the nebulizer mask over their own nose and mouth. In some embodiments, where the mask lacks strap attachments (e.g., the mask 600 described with reference to FIGS. 6A, 6B, and 6C), the user may manually hold the mask in place. At block 706, the patient or another user may activate the nebulizer to generate an aerosolized mist. This may involve pressing a power button or initiating airflow through a mechanical or electronic activation system. The nebulizer converts the liquid medication into fine particles that are directed into the mask’s interior chamber.

At block 708, the patient inhales the aerosolized medication through the nebulizer mask. As the patient breathes in, ambient air enters through the perforations and mixes with the aerosolized medication. The structured perforation pattern allows for controlled air mixing, optimizing the concentration of medication delivered with each breath. The patient may continue breathing through the mask for the prescribed duration. The duration may vary depending on the medication and treatment plan. In some examples, the user may adjust the mask’s perforations, if they are configurable, to modify airflow resistance or medication concentration.

At block 710, after the treatment is completed, the patient or another user removes the mask from the patient’s face and powers off the nebulizer. If necessary, the user may wipe condensation from the mask’s interior and inspect the nebulizer for remaining medication. The patient may then clean and store the nebulizer mask and device. This may involve rinsing the mask with warm water or using a manufacturer-recommended cleaning solution to maintain hygiene. In some examples, masks with removable perforation covers or filters may require additional disassembly for thorough cleaning. The nebulizer system is then stored in a dry, safe location for future use.

As discussed, aspects of the present disclosure are directed to a nebulizer mask that improves the delivery of aerosolized medication by incorporating a structured arrangement of perforations that allow controlled ambient air mixing. Various examples of the nebulizer mask include different perforation patterns, connector interfaces, and structural configurations that optimize airflow, medication retention, and user comfort. The nebulizer mask may be configured to work with standard nebulizer systems, ensuring compatibility across different medical and home-use applications.

In some examples, the nebulizer mask includes a group of perforations distributed across its surface, allowing ambient air to enter the interior chamber and mix with the aerosolized medication before inhalation. These perforations may be circular openings, elongated slits, or larger holes positioned on the lateral sides of the mask. In one example, the perforations are symmetrically arranged, with a first perforation running along a center line and additional perforations positioned at an angle relative to the first perforation. In another example, the perforations may be uniform in size, while in yet another, they may gradually decrease in length as they extend outward from the center. In some examples, the perforations collectively cover at least ten percent of the total surface area of the mask to allow sufficient ambient air mixing while maintaining medication concentration. Depending on the intended use of the mask and the required balance between airflow and medication retention other surface area coverages are contemplated.

The nebulizer mask may include various connector interfaces for attachment to a nebulizer device. In one example, the connector interface is cylindrical and designed for a friction-fit connection, while in another example, it includes a threaded or locking mechanism for enhanced stability. Some examples of the connector interface feature internal ridges, gaskets, or sealing rings to prevent leaks and improve the attachment between the mask and the nebulizer. The mask’s tip may be flat to provide a stable transition between the mask body and the connector interface, ensuring consistent airflow through the mask.

The structure of the nebulizer mask may also vary depending on user needs. In some examples, the mask includes tabs at its lateral ends, which provide attachment points for securing straps. In other examples, the mask does not include tabs and instead features a continuous outer perimeter designed to fit snugly against the user’s face. The material composition of the mask may include flexible medical-grade materials such as silicone, PVC, or thermoplastic elastomers, providing a balance between durability and comfort.

Some examples of the nebulizer mask include adjustable perforations that allow users to regulate the amount of ambient air mixing. These perforations may feature sliding covers, adjustable membranes, or one-way airflow valves that permit air entry while preventing medication loss during exhalation. In other examples, the perforations may be fixed but positioned asymmetrically to create directional airflow patterns that optimize the dispersion of aerosolized medication within the mask. In certain examples, the perforation surface area may be selectively reduced by using removable inserts, allowing the user or caregiver to adjust airflow resistance according to treatment needs.

The nebulizer mask is intended for use in a variety of respiratory treatments, including the administration of bronchodilators, corticosteroids, and saline solutions. The structured perforation pattern reduces inhalation resistance and minimizes condensation buildup, preventing medication waste. The mask’s design also improves breathability, making it more comfortable for extended treatments.

As used herein, the term “determining” encompasses a wide variety of actions. For example, “determining” may include calculating, computing, processing, deriving, investigating, looking up (e.g., looking up in a table, a database or another data structure), ascertaining and the like. Additionally, “determining” may include receiving (e.g., receiving information), accessing (e.g., accessing data in a memory) and the like. Furthermore, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

It is to be understood that the claims are not limited to the precise configuration and components illustrated above. Various modifications, changes, and variations may be made in the arrangement, operation, and details of the methods and apparatus described above without departing from the scope of the claims.

What is claimed is:

1. A nebulizer mask, comprising:
a body defining an interior chamber for receiving aerosolized medication, the body having an interior surface and an exterior surface;
a connector interface, at a tip of the mask, for attaching the nebulizer mask to a nebulizer device; and
a group of perforations on the body, each perforation penetrating the interior surface and the exterior surface.

2. The nebulizer mask of claim 1, wherein the group of perforations collectively cover at least ten percent of a surface area of the mask.

3. The nebulizer mask of claim 1, wherein each perforation of the group of perforations is a circular opening.

4. The nebulizer mask of claim 1, wherein each perforation of the group of perforations include is an elongated slit.

5. The nebulizer mask of claim 1, wherein the group of perforations include a first set of perforations and a second set of perforations, the first and second set of perforations are symmetrically arranged on opposite sides of the mask.

6. The nebulizer mask of claim 5, wherein:
a respective first perforation of each of the first and second set of perforations is positioned along a center horizontal axis of the mask; and
one or more additional perforations extend at an angle relative to center horizontal axis.

7. The nebulizer mask of claim 5, wherein:
each perforation of the group of perforations include is a circular opening;
a respective first subset of perforations of each of the first and second set of perforations is positioned along a center horizontal axis of the mask;
one or more additional subsets of perforations are defined along respective horizontal axes; and
each one of the additional subsets of perforations is parallel to center horizontal axis.

8. The nebulizer mask of claim 1, wherein a height of each perforation of the group of perforations is uniform.

9. The nebulizer mask of claim 1, wherein a height of each perforation of the group of perforations tapers from the tip of the mask to an outer perimeter of the mask.

10. The nebulizer mask of claim 1, wherein the connector interface includes a friction-fit mechanism, a threaded attachment for securing the mask to the nebulizer device, or a locking mechanism to prevent unintended detachment from the nebulizer device.

11. The nebulizer mask of claim 1, wherein the mask comprises silicone, PVC, or thermoplastic elastomers.

12. The nebulizer mask of claim 1, wherein the mask covers a nose and mouth of a user.

13. A method for delivering aerosolized medication using a nebulizer mask, comprising:
attaching the nebulizer mask to a nebulizer device;
directing the aerosolized mist into an interior chamber of a nebulizer mask, the mask having a group of perforations for allowing mixing of the aerosolized mist with ambient air; and
inhaling the aerosolized mist through the nebulizer mask, the group of perforations regulating airflow and medication concentration.

14. The method of claim 13, further comprising exhaling through the nebulizer mask, wherein the group of perforations provide passive airflow.

15. The method of claim 13, wherein the group of perforations collectively cover at least ten percent of a surface area of the nebulizer mask.

16. The method of claim 13, wherein:
the group of perforations include a first set of perforations and a second set of perforations symmetrically arranged on opposite sides of the mask; and
each one of the first and second set of perforations includes a first perforation defined along a center horizontal axis of the nebulizer mask and one or more additional perforations extending at an angle relative to the center horizontal axis.

17. The method of claim 13, wherein the group of perforations include:
a first subset of perforations positioned along a center horizontal axis of the mask; and
one or more additional subsets of perforations positioned along respective horizontal axes that are parallel to the center horizontal axis.

18. The method of claim 13, wherein:
the nebulizer mask comprises a connector interface at a tip of the nebulizer mask; and
the connector interface being configured to attach the nebulizer mask to the nebulizer device via a friction-fit mechanism, a threaded attachment, or a locking mechanism.

19. The method of claim 13, wherein the nebulizer mask is placed over a nose and mouth of a user to facilitate respiratory therapy.

20. The method of claim 13, wherein each perforation of the group of perforations is a circular opening.
